(19) United States
(12) Patent Application Publication
(10) Pub. No.: US 2005/0208593 A1
(43) Pub. Date: Sep. 22, 2005

(54) Lateral flow diagnostic assay reader with radial cassette

(75) Inventors: Timothy L. Vail, Parks, AZ (US); Jerry M. Hatfield, Flagstaff, AZ (US); Catherine Propper, Flagstaff, AZ (US)

Correspondence Address: JENNINGS, STROUSS & SALMON, P.L.C., 201 E. WASHINGTON ST., 11TH FLOOR, PHOENIX, AZ, 85004, US

(73) Assignee: Arizona Board of Regents, acting for and on behalf of Northern Arizona University

(21) Appl. No.: 11/085,717

(22) Filed: Mar. 21, 2005

Related U.S. Application Data
(60) Provisional application No. 60/554,855, filed on Mar. 19, 2004.

(51) Int. Cl. C12Q 1/68; G01N 33/53; C12M 1/34

(52) U.S. Cl. 435/7.1; 435/287.2

(57) ABSTRACT

The present invention provides a method and field portable apparatus for analyte detection using lateral flow immunochromatographic assays, where a single test sample may be simultaneously applied to multiple assays.

### CROSS-REFERENCE TO RELATED APPLICATIONS

This application claims the benefit of priority of U.S. Patent Application Ser. No. 60/554,855 filed Mar. 19, 2004.

### FIELD OF THE INVENTION

The present invention relates to lateral flow assays, particularly immunoassays. More specifically, the invention provides a method and apparatus for determining the amount of an analyte present in a sample.

### BACKGROUND OF THE INVENTION

Accurate methods for analyzing water and soil quality have become increasingly important in the prevention of pathogen-borne diseases and chemical contamination, particularly in arid areas where dilution is not an option, and where the use of reclaimed wastewater appears to be a viable option for conservation, irrigation, and recreational purposes.

Many anthropogenic compounds are constantly released into the environment, including pesticides, agricultural hormones, pharmaceuticals, household chemicals, and industrial compounds. One of the largest sources of these compounds comes from wastewater release. There is strong evidence that many of these compounds are found at biologically relevant concentrations in wastewater and can pose a hazard to both humans and animals.

In particular, steroid hormones present in the environment have the potential to affect the health of animals and humans. For example, estrogenic compounds including the active ingredient of oral contraception, ethynyl estradiol, are found in wastewater at concentrations that are known to have estrogenic effects in fishes, and have shown up in drinking water at levels that could be considered biologically active. Municipal water effluent may also contain hundreds of compounds, including the active ingredients in common over-the-counter drugs, insect repellant, caffeine, tobacco by-products, and other commonly ingested or topically applied agents. The effects of low concentrations of these hormonally active chemicals, personal care products, and pharmaceuticals is unknown, but these compounds have the potential to cause a physiological response in humans, plants, and animals that come into contact with these compounds in wastewater or in municipal water supplies.

In addition, accurate methods to qualitatively and quantitatively measure pharmaceutical or nutritional compounds (e.g., pharmaceuticals produced in foreign countries) are important for quality control and safety screening purposes. Further, there is a need for assays to detect pathogens present in environmental samples and to measure the presence of pathogens, hormones, or other contaminants present in a biological sample.

One recent method of detection of analytes in environmental or biological samples is immunochemical technology. Immunochemical technology has been applied to environmental analysis using a variety of assay techniques and platforms in order to detect these contaminants.

Broadly defined, immunochemical technology includes any methodology that exploits the binding characteristics of antibodies (typically by utilizing labeled probes), either directly or indirectly coupled to a chemical, enzyme, or particle (called reporters). The reporter functions by “reporting” the presence of the object of interest (the analyte) such as a chemical toxin, virus, bacterium, nucleic acid sequence, hormone, and the like, through the use of a detector. The reporter component is often coupled directly to the probe, such as an antibody protein covalently attached to a fluorescent molecule. This probe-reporter pair is therefore intended to provide a signal that is proportionally related to, either quantitatively or qualitatively, the concentration of analyte in the sample. Detecting the presence of the reporter (and therefore the analyte) is usually accomplished by means of a peripheral device such as a spectrophotometer, sub-atomic particle detector, or by direct visual inspection.

The critical characteristics and limiting factors of immunochemical technology include the necessity for a probe that recognizes the target analyte with a high degree of specificity, a temporally and chemically stable reporter that gives a signal proportionally related to the presence of an analyte, and a detection system capable of sensing very low levels of the reporter signal, and then relaying information from the reporter to a method of interpretation.

A variety of immunoassay techniques for determining the presence or amount of an analyte in a sample have been available for many years. Known devices in the lateral flow technology area typically involve a system wherein one end of a carrier-held test strip is exposed to the sample being tested for a given target analyte, and the result is read visually (e.g., home pregnancy tests, ketosis testing strips).

Immunoassay technology presently allows for assay tests to be performed without the need for expensive, cumbersome equipment or instrumentation generally found in an institutional laboratory setting. However, a limitation exists in that operator subjectivity in reading and interpreting results is often problematic. In addition, accuracy may be compromised as the assay strip is increasingly subjected to contamination by exposure to light degradation or other elements.

A further limitation in the known art is the lack of the ability to simultaneously process more than only a few samples, often requiring multiple applications of the sample to numerous test pad areas.

Therefore a need exists for a system and method that provides a rapid, sensitive, and portable diagnostic assay platform for determining the amount of an analyte present in a sample, based on polyclonal antibodies specifically designed to detect known analytes in a sample, including pharmaceuticals, hormones, consumer products, industrial compounds and agricultural pesticides.

### BRIEF DESCRIPTION OF THE INVENTION

The present invention provides a method and hand-held apparatus for performing multiple assays, particularly in the field, for use in monitoring environmental contaminants, or in a small laboratory setting for quantitatively and qualitatively analyzing the results of a lateral flow assay to a high degree of sensitivity and specificity.

### BRIEF DESCRIPTION OF THE DRAWINGS

FIG. 1 is a schematic of the top view of the cassette unit in the diagnostic assay reader.

FIG. 2 is a schematic of the top view of the cassette unit.

FIG. 3 is a schematic of an alternative reader configuration.

FIG. 4 is a schematic of the side view and top view of the assay strip.

FIG. 5 is a schematic top view of one embodiment of a cassette.

FIG. 6 is a side view of the cassette of FIG. 5.

FIG. 7 is a side view of an alternative embodiment of a cassette.

FIG. 8 is a top view of a sample flow distributor.

FIG. 9 is a side view of the flow distributor of FIG. 8.

FIG. 10 is an alternative version of a cassette for holding multiple assay strips.

FIG. 11 is a functional block diagram of an assay reader.

FIG. 12 is a cross sectional view of a version of an assay reader.

### DETAILED DESCRIPTION OF THE INVENTION

The present invention is directed to an apparatus and method for portable testing for pollutants, contaminants or the like by determining the amount of analyte associated with the targeted substance present in a sample. More specifically, the present invention is directed to both a cassette device and optical scanner for the quantitative or semi-quantitative analysis of lateral flow immunochromatographic diagnostic assays.

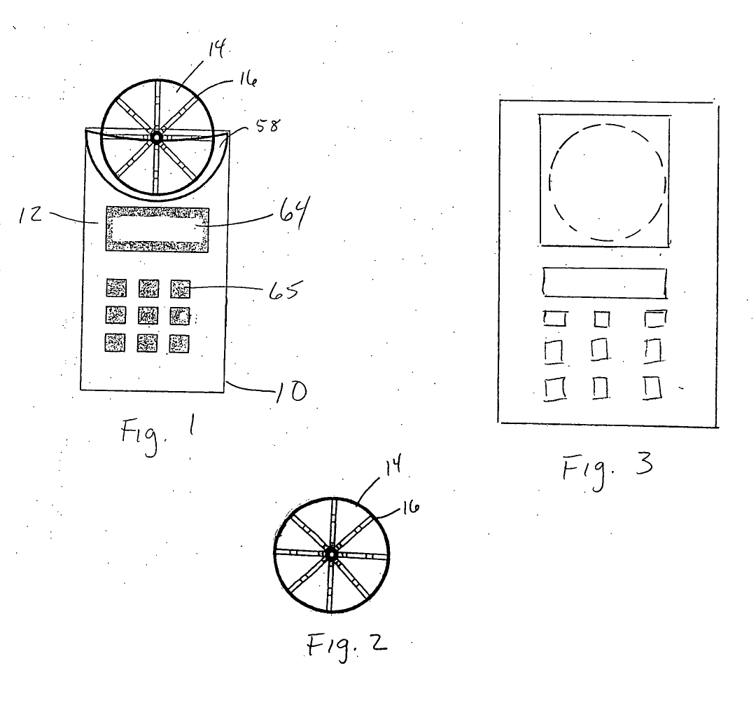

In multiple embodiments of the invention, the lateral flow diagnostic assay reader 10 (FIG. 1) is field-portable, and comprises a spectrophotometeric scanner 12, a replaceable cassette unit 14 (FIGS. 1, 2), capable of receiving a plurality of immunochromatographic assay strips 16, coupled to an electronic data storage unit and a retrieval interface.

Assay Strips

In the preferred embodiment, the assay strips 16 of the present invention are disposable and are further capable of being releaseably inserted into the cassette unit 14. The cassette unit 14 is capable of receiving one or a plurality of assay strips 16, depending on the number of samples to be analyzed.

In one embodiment of the invention (FIG. 4), the assay strips 16, have a solid support substrate 32, which may be rigid or semi-rigid. This substrate 32 is made of a nitrocellulose layer 34; however, it is within the scope of the present invention that the layer 34 is any material that is an insoluble matrix, including blotting material, capillaries, cellulose, silica, polystyrene, latex, or glass coated with a polymer.

The nitrocellulose layer 34 is a part of a lateral flow system, wherein a first absorbent filter pad 40 is applied to the substrate 32, at proximal end of the assay strip 16, wherein the lateral flow system is capable of wicking a sample from one end to the other across the assay strip. In this embodiment of the invention the first absorbent filter pad 40 is capable of receiving a sample, filtering out any large particulate matter in the sample, and holding the sample so that it can slowly wick into the assay.

In the preferred embodiment of the invention, the conjugate 42 is bound to the substrate 32 of the assay strip 16. In another embodiment, the first absorbent filter pad 40 is further capable of receiving a conjugate 42, comprised of probe-reporter pairs 44, and the conjugate 42 is applied to the first absorbent filter pad 40. The conjugate 42 is comprised of assay reporter 36 bound to a detector 38 antibody; however, it is within the scope of the invention that the assay reporter 36 may be associated with any molecule with an affinity to a target analyte, including molecules that can bind to antigens, proteins, nucleic acids, cells, sub-cellular organelles, and other biological molecules. Alternatively, the assay may be set up as a competitive design where the assay reporter 36 is conjugated to a known amount of analyte and captured via an antibody or other compound with a specific affinity for the analyte.

In the preferred embodiment, the probe-reporter pairs 44 are comprised of a plurality of pairs of a detector antibody 38 bound to an assay reporter 36. In this embodiment, the detector antibody 38 is specific to at least one target detectible compound. The probe-reporter pair 44 is dried down into the first absorbent filter pad 40.

In the preferred embodiment, the assay reporter 36 is a fluorescent semi-conducting nanocrystal also known as a quantum dot (including QDots®, available from Quantum Dot Corp., Hayward, Calif., and EviTag® Quantum Dots, available from Evident Technologies, Troy, N.Y.). These quantum dots are known to exhibit the following properties: resistance to damage or change in properties due to environmental factors such as pH and temperature, resistance to photochemical alteration during excitation and emission (no photo-bleaching), capable of ready coupling to commercially available antibodies, and capable of easily drying down on a test strip for immunochromatography. Further, the quantum dots have the following properties: (i) high fluorescent intensity (for detection in small quantities), (ii) a separation of at least 50 nm between the absorption and fluorescing frequencies, (iii) solubility in water, (iv) ability to be readily linked to other molecules, (v) stability towards harsh conditions and high temperatures, (vi) a symmetric, nearly Gaussian emission lineshape for easy deconvolution of multiple colors, and (vii) compatibility with automated analysis.

In the preferred embodiment, the assay reporter 36 can detect the presence or amounts of a biological or chemical moiety; the structure, composition, and conformation of a biological moiety; the localization of a biological or chemical moiety in an environment; interactions of biological or chemical moieties; alterations in structures of biological or chemical compounds; and alterations in biological or chemical processes. In the preferred embodiment, the assay reporter 36 has a characteristic spectral emission, which is tunable to a desired energy by selection of the particle size of the quantum dot and an affinity for a target analyte. In this embodiment, the location and nature of the association can be detected by monitoring the emission of the assay reporter 36.

In the preferred embodiment, the assay reporter 36 has a range of excitation wavelength that is broad and allows the simultaneous excitation of all assay reporters 36 in a system with a single light source, preferably in the ultraviolet or blue region of the spectrum, and is resistant to degradation or photobleaching over time.

Additionally, within other alternate embodiments of the present invention, the conjugate 42 comprises standard assay reporters 36 (e.g., labeled colloidal gold, latex beads, lanthanide-doped ceramic nanoparticles) that are colloidally stabilized, highly sensitive, small diameter particles with high surface area/volume ratios capable of attachment of biological ligands, and bound to the detector antibody 38 to form the probe-reporter pair 44. More specifically, the detector 38 may be derived from polyclonal or monoclonal antibody preparations, may be a human antibody, or may be a hybrid or chimeric antibody, such as a humanized antibody, an altered antibody, F(ab′)2 fragments, F(ab) fragments, Fv fragments, a single-domain antibody, a dimeric or trimeric antibody fragment construct, a minibody, or functional fragments thereof which bind to the analyte of interest. Antibodies are produced using techniques well known to those of skill in the art. Furthermore, the detector 38 may be a receptor for a specific hormone or any other protein with specific binding affinities.

The assay strip 16 (FIG. 4) further comprises a capture line 46, and a read line 48, two spatially distinct zones, applied and dried onto the substrate 32 of the assay strip 16. The capture line 46, is comprised of capture molecules 37 bound to the assay strip 16, which are capable of “capturing” antigens present in the sample, via antigen-antibody affinity binding, when the sample flows past the capture line 46. In the preferred embodiment, the capture molecules 37 are a known concentration of the specific target analyte, further capable of capturing reporter-probe pairs 44 that are not bound to analyte present in the sample. Accordingly, the fluorescence emitted at the capture line 46 will decline with increasing amounts of analyte present in the sample.

The read line 48 comprises a known concentration of commercially available anti-species antibody antibodies 39. Anti-species antibodies 39 are capable of binding with the reporter-probe pair 44 whether antigens are present or not, and serve to indicate whether the assay is functioning properly, and may be proportional to the amount of reporter-probe pairs 44 bound to the analyte in the sample, providing another level of sensitivity to the assay.

The assay strip 16 further comprises a second absorbent filter pad 50, located at the distal end of the assay strip 16. The second absorbent filter pad 50 is capable of absorbing and holding the sample after it has wicked across the assay strip, preventing the sample from flowing in the opposite direction, and causing non-specific binding to occur.

When a sample is applied to the first absorbent filter pad 40, the sample laterally flows toward the second absorbent filter pad 50, washing over the capture line 46 and read line 48. The application of negative sample (˜0% concentration of the target analyte) results in all (or nearly all) of the conjugate 42 of probe-reporter pairs 44 present on the capture line 46 to bind, via antigen-antibody binding, with the analyte present in the sample, and little or no binding with the antibodies 39 present at the read line 48 (FIG. 4). Conversely, when analyte is present in the sample, the free analyte binds to the detector antibody 38 in the probe-reporter pair 44, preventing the probe-reporter pair 44 from binding at the capture line 46. The probe-reporter pair 44 passes through the capture line 46 and is bound by the antibodies 39 at the read line 48. With a lot of analyte present, the capture line 46 will have low binding and the read line 48 will have high binding with the probe-reporter pairs 44. As the amount of analyte declines, more of the probe-reporter pairs 44 are free to bind to the absorbed constant amount of analyte on the capture line 46 and less is available on the read line (although there will always be some bound to the read line as the amount of probe-reporter pairs 44 will always be in excess of what can bind to the capture line 46). Excess sample will be wicked into a second absorbent filter pad 50 at the distal end of the assay strip 16.

In multiple embodiments of the invention, the assay strips 16 also include a reference spot 52, comprising a known pre-measured concentration of probe-reporter pairs 44 spotted and dried down to a unique known location, onto the substrate 32 of the assay strip. The reference spot 52 may be any shape or size, including a circular spot or a line. The known pre-measured concentration of probe-reporter pairs 44 at the reference spot 52 will produce a fluorescence reading, which serves as a control, allowing the reader to detect the quantity of fluorescence produced by the reference spot 52. The reference spot 52 is further capable of providing calibration for the instrument, and comparing assay test results to this control. Use of the reference spot 52 further allows automatic calibration of the diagnostic assay reader in the field.

It is further contemplated that the assay strip 16 may alternatively use traditional assay reporters 36 for detecting analytes including radioactive markers; fluorescent molecules as tags including mono or polyclonal antibodies labeled with a fluorescent tag (e.g., fluorescein, propidium iodide, Hoechst dyes, ethidium bromide, methyl coumarin, or Texas red) and directed at a particular target; and secondary antibodies tagged with a fluorescent marker and directed to the primary antibodies to visualize the target.

### EXAMPLE 1

### Assay Strip Configuration

In one example, the assay strip 16, was configured using Ethynyl Estradiol (EE2) as the analyte. The assay was set up as a “competitive” assay, such that the amount of analyte present in the sample will be inversely proportional to the signal detected. The overall dimensions of the assay strips 16 were approximately 0.6 cm×6.0 cm.

The assay was designed as follows: the conjugate 42 comprised a commercially available assay reporter 36, Protein A-conjugated Quantum Dots (Quantum Dot Inc., Hayward, Calif.). These assay reporters 36 have fluorescent emission in the visible range (AEX=360 nm; AEM=605 nm), and were incubated with saturating concentrations of mouse monoclonal antibodies specific for Ethynyl Estradiol. Since Protein A binds the Fc (non-binding) portion of antibodies 38 with high specificity, this resulted in a probe (antibody)-reporter (QD) pair that is specific and sensitive for EE2.

This conjugate 42 was then dried into the first absorbent filter pad 40 comprised of Millipore Corp. application fleece (Millipore Corp., Bedford, Mass.). The substrate 32 is comprised of mylar-backed nitrocellulose (Millipore Corp., Bedford, Mass.). Downstream from the first absorbent filter pad 40, two spatially distinct zones were applied and dried onto the strip. The capture line 46 comprised a known concentration (optimized through testing) of EE2. The second zone, the read line 48, comprised a known concentration (optimized through testing) of commercially available goat anti-mouse antibody.

Sample volumes in the 100-200 uL range were applied to the assay strips. The application of a negative sample (0% concentration EE2) resulted in nearly all of the QD-mouse anti-EE2 binding to the EE2 on the capture line 46, with low QD binding at the read line 48. When EE2 was present in the sample, the free EE2 bound to the QD-antibody conjugate, preventing the conjugate from binding at the capture line. When the sample flowed through the capture line, it bound to the anti-mouse antibodies 38 at the read line 48. Excess sample was then wicked into the second absorbent filter pad 50 at the distal end of the assay strip 16. The strip was the ready to be scanned using the diagnostic assay reader 10.

### EXAMPLE 2

### Assay Strip Analytes

Assay strips 16 will be designed as sensitive and specific immunochemical tests for each of the analytes in Table 1.

TABLE 1
Proposed Compounds for Assay Development

| | Industrial | |
|---|---|---|
| Pharmaceuticals | Intermediates | Pesticides |
| Ethynyl Estradiol | Bisphenol A | Diazinon |
| Equilin | 4-Nonylphenol | Carbaryl |
| Carbamazebine | 4-Nonylphenol | Endosulfan |
| Estradiol | monoethoxylate | |
| Testosterone | 4-tert Octyl phenol | |
| | 4-tert Octylphenol | |
| | monoethoxylate | |
| | Galaxolide | |

Cassette Unit

The cassette unit holds the assay strips for application of the sample and for scanning of the assay strips inside the reader. The cassette unit may be a disposable single-use device or re-loadable so that assay strips may be replaced after use. In a preferred embodiment of the invention (FIGS. 5, 6), the cassette unit 14 is a circular disc 22, with a hub 24 as an axial pivot point, and with attached assay strips 16 extending radially from the hub 24 in a spoke-like configuration.

In this embodiment, the spokes 16 outwardly connect from a central sample port 28 at hub 24. The cassette unit design is such that each assay strip 16 is mounted in a fashion that allows each assay to flow laterally from the hub 24, outward toward the perimeter 20 of the cassette unit. A downwardly sloping surface 25 from the hub 24 to the perimeter 20 may be employed to encourage the flow, but capillary wicking through the substrate will remain the primary mechanism. In one embodiment of the invention, each cassette unit 14 holds a plurality of fixedly-mounted single-use assay strips 16. The cassette units 14 are removable from the diagnostic assay reader 10, and may be archived or disposed of. In another embodiment of the invention, the cassette unit 14 is reusable and is capable of receiving new single-use assay strips 16 after processed ones are removed.

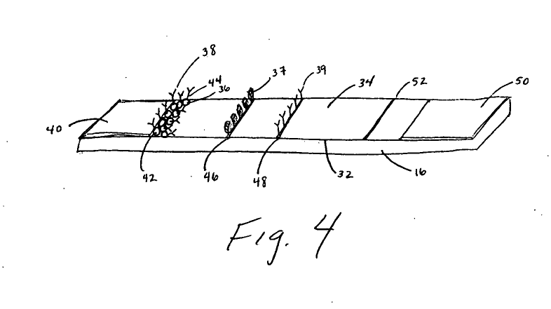

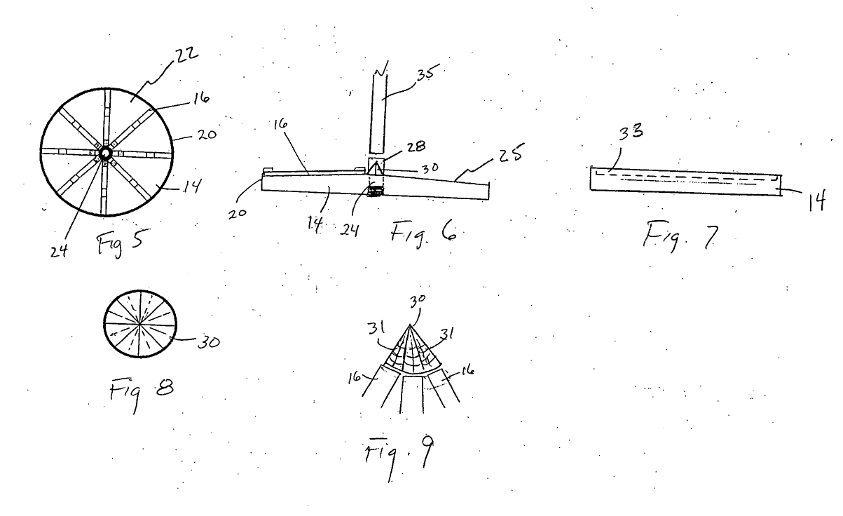

The cassette unit may be fabricated of any lightweight rigid material, such as metal, polystyrene, polycarbonate, or similar durable plastic. Some method of attaching assay strips to the cassette must be provided. In one embodiment (FIG. 7), appropriately-sized shallow slots 33 may be formed in the cassette unit 14 surface, into which assay strips 16 may be inserted. In another embodiment, the assay strips 16 may be manufactured with an adhesive backing on the bottom surface, allowing them to be stuck onto the cassette unit 14 disc.

If the cassette unit 14 is to be used with a reader unit that provides for an excitation source 71 positioned beneath the assay strip 16 (FIG. 12), then the cassette unit must be transparent to the excitation energy, at least directly under the assay strip. For example, the cassette unit 14 material under the slots 33 in FIG. 7 might be clear plastic. Alternatively, the entire cassette unit 14 body may be clear plastic. In addition, the excitation source 71 may be positioned above the assay strips 16 with a control for turning it off prior to reading the emission.

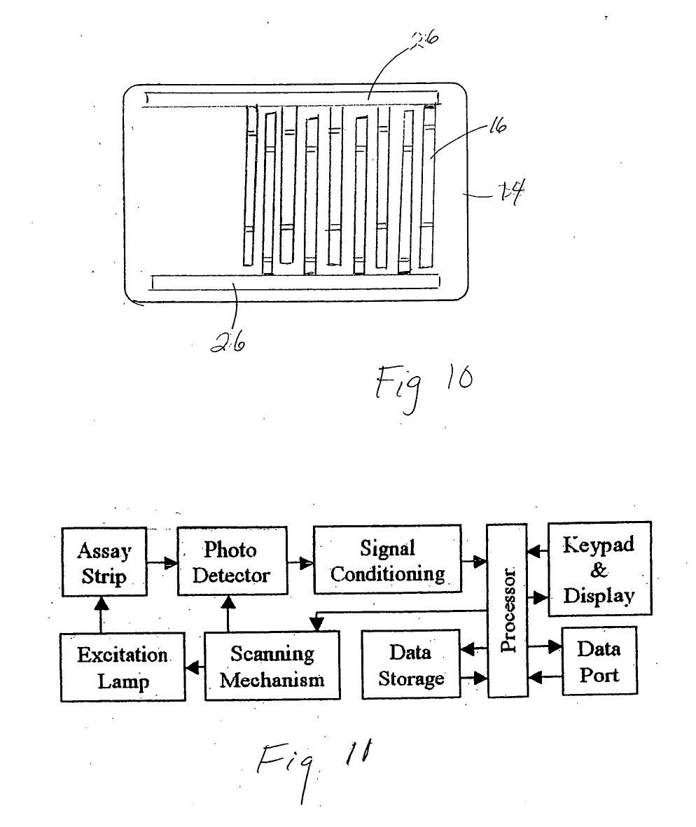

In a preferred embodiment, (FIGS. 8-9) the cassette unit 14 is designed to receive a water sample from a pipette 35 in which the sample is taken or stored. The pipette mates to the sample port 28, and the water is injected. The sample is subdivided or split simultaneously upon application by a sample distributor 30, such that pre-determined volumes are selectively directed to a plurality of assay strips 16. The assay strips 16 may each be for a single specific analyte or some redundancy may be employed. In this embodiment of the invention, a sample distributor 30 comprises a cone configured with a plurality of channels 31 down the outer surface of the cone to distribute a portion of the sample into each assay strip 16. The distributor 30 may channel an equal amount of the sample to each of the radiating assay strips 16, or it may, where warranted, divide and direct unequal amounts of sample to specific assay strips 16.

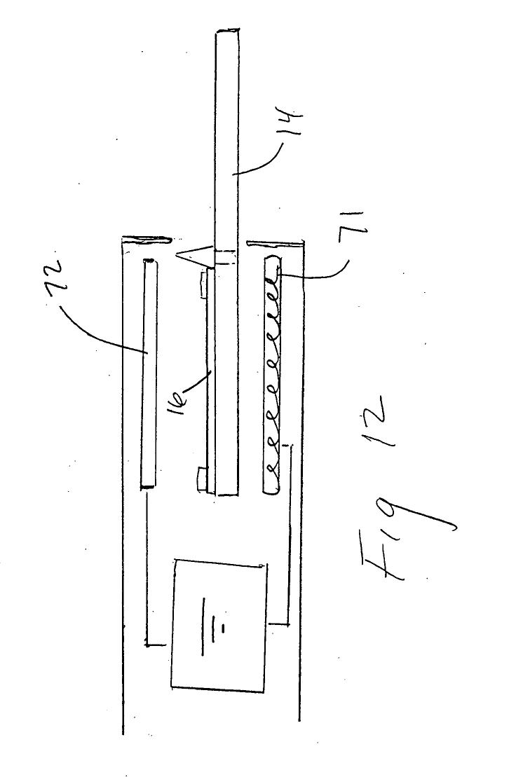

Cassette unit 14 configurations other than the disc with spokes may also be used. For example, a parallel slot configuration may be desirable in some applications; see FIG. 10. The sample could be flowed into a manifold trough 26 for distribution to the assay strips, or samples could be applied to the proximal pad directly from a dropper or other device. Configurations of any alternative cassette would have to match the configuration of the reader with which it is used.

Diagnostic Assay Reader

As shown in one embodiment in FIG. 1, the diagnostic assay reader 10 is a hand-held unit configured to accept a cassette unit 14, containing a plurality of assay strips 16, via a slot 58. The diagnostic assay reader 10 is capable of reading assays, recording results, and archiving multiple results for later retrieval and analysis. A functional diagram of the assay reader is shown in FIG. 11.

In yet another embodiment, diagnostic assay reader 10 comprises a portable, closeable instrument case configuration. See FIG. 3. In this embodiment, it is further envisioned that this configuration includes one or a plurality of features including: protective case, battery storage, accommodation for larger batteries, storage for additional cassette units and storage for additional array strips.

In at least one embodiment as shown in FIG. 1, the diagnostic assay reader 10 includes a screen display 64 and memory capable of receiving user-entered data (e.g., location site name, latitude, longitude from separate GPS, weather conditions, date and time) using an interface similar to a Personal Data Assistant. User data may be entered via a touch screen or a keypad 65.

The diagnostic assay reader must provide an excitation source 71 and an emission receptor 72 (FIG. 12) to extract information from the assay strips 16, and wherein the excitation source is a source that provides the appropriate range of excitation based on the type of assay reporters 36 used in the assay. In one embodiment, the excitation source 71 is an ultraviolet light source, such as a xenon lamp, positioned in the reader case below the assay strip being processed. Positioned above the assay strip is a photodiode detector array emission receptor 72, which senses the fluorescent energy radiated by the conjugates on the strip in the predetermined wavelength and maps the intensity distribution along the length and across the width of the strip. The intensity distribution is then stored or displayed on both.

With a system comprising a reader matched to a circular cassette with radial alignment of assay strips, a strip may be aligned under the detector array and reading taken by activating the excitation source 71 and emission receptor 72. Once the reading is complete, the cassette unit 14 may be rotated until the next strip aligns with the detector and that assay strip 16 may be read.

In another embodiment of the reader the spectrophotometeric scanner comprises a fiber-optic raster, with both excitation and emission sources. The excitation source is an excitation lamp, comprising a light source (which may include, but is not limited to a UV light sources, such as xenon lamp or other light source appropriate to the type of assay reporters 36 used in the assay) coupled to a fiber optic line such that the excitation lamp beam can be moved electromechanically over the entire length of the assay strip 16, after the assay has run to completion. The excitation lamp beam further comprises an optical fiber in close proximity to an emission detection fiber bundle, which captures emission light from assay components, namely chromatophores including fluorescent molecules, fluorescent sub-micron particles including fluorescent latex, quantum dots, up-converting and down-converting phosphors complexed to biological probe reagents such as antibodies and nucleic acids.

The excitation lamp beam and emission detection fiber bundle are moved by the scanning mechanism along the length of each assay strip 16 to collect fluorescent emission values as a function of distance along the strip relative to the capture line 46 and read line 48.

The fiber-optic raster is coupled to a photo-detector, wherein the emitted light levels are optically ported to the photo-detector. The photo-detector is a photodiode linear imaging array, such as a photomultiplier tube, charge-coupled device or similar, wherein the photo-detector is capable of determining light levels as a function of location on the assay.

The photo-detector is further capable of spanning the width of the assay strip 16 and provides a cross-strip measurement of emission values at each location along the length of the assay strip 16. In an alternative embodiment of the invention, the excitation lamp beam is in a fixed position, and the emission detection fiber bundle will pick up the light signal emitted by the excited fluorophore and carry it to the photo-detector. In this embodiment, both the excitation lamp beam and emission detection fiber bundle would not travel along the assay strip 16, only the ends of the emission detection fiber bundle would travel along the assay strip 16.

Reading the Assay Strips with the Reader

In a preferred embodiment, the invention utilizes electro-optical scanning of the assay strip 16 scanning in order to reduce the complexity and increase the reliability and ruggedness of the diagnostic assay reader 10. However, in an alternative embodiment of the invention, the assay strip 16 may also be scanned mechanically.

When a user is ready to read the assay strips 16, a cassette unit 14, filled with one or a plurality of assay strips 16, must be inserted into the slot 58 in the diagnostic assay reader 10. In order for the diagnostic assay reader 10 to read an assay strip 16, the diagnostic assay reader 10 must first receive a designated signal from the reference spot 52 as an internal diagnostic control. Reference spots 52 are integrated into the assay strip 16 or cassette unit 14 for each assay in order to function as an internal instrument check and as a signal comparator or reference to which the assay signal will be compared. In this embodiment, because the diagnostic assay reader is intended to be field-operated under varying conditions of temperature and relative humidity, the reference spot 52 provides a relative standard (subject to the same ambient conditions) as the test signal.

In this embodiment, once the reference spot 52 is read, this signal will be stored in the electronic data storage unit and compared to the signal from the read line 48 and capture line 46 on the assay strip 16. During the course of assay development for specific analytes, signal/concentration data are determined for each assay read line 48 and capture line 46 and programmed into the electronic data storage unit for comparison to the reference spot 52. In this embodiment, the diagnostic assay reader 10 detects the fluorescence emitted from the read line 48 and the capture line 46, these fluoresce readings are directly proportional to the concentration of analyte in the test sample.

In another embodiment of the invention, data is reported as fluorescence intensity, or brightness. In addition, the data may be collected and interpreted based on the function of horizontal distance along the test strip, or the distance of migration of the probe-reporter pairs 44 along the assay strip 16, using techniques and analysis similar to gel electrophoresis. In this embodiment, the probe-reporter pairs 44 would move along the strip to different distances depending on whether or not they were bound to the analyte. The proportion of the intensity of the assay reporter 36 signal between the two lines would be an indirect measure of analyte in the sample.

Data Analysis

Assay data may then be generated as an emission intensity profile, allowing the instrument to compare the fluorescence intensities of the capture line 46, read line 48, and reference spot 52, in the determination of analyte concentration. In at least one embodiment of the invention, based on these intensities, an algorithm, such as a direct ratio, or a slope determination which varies inversely with sample concentration, is used to determine the analyte concentration present in the sample.

This algorithm is used to generate a quantitative or semi-quantitative result (for example the detection of a particular analyte displayed on the readout in parts per million or billion). Further, this data point is stored such that the user inputs unique alphanumeric identifiers associated with the data point (such as location name, global positioning system coordinates, or other unique identifiers). The electronic data storage unit allows the user to recall the data on screen display 64 and also to upload the data via wired or wireless connection to a personal computer for the creation of a database.

Several signal comparison algorithms may be executed during the operation of the diagnostic assay reader 10. One such algorithm is a signal comparison between signals emitted by the sample and a pre-applied control reference spot 52 for each assay that is integrated into the cassette. The reference spot 52 provides an emitted light signal at a unique address (location) on the strip. This address may be programmed into the diagnostic assay reader 10.

Before scanning the length of an assay strip 16, the diagnostic assay reader 10 first moves to the reference spot 52 to receive a start signal. The diagnostic assay reader 10 must receive a positive threshold signal value from the reference spot 52. If this signal is not received, or is below an assigned threshold value, the diagnostic assay reader 10 does not continue with the reading of the assay strip 16. Instead, it enters an “error mode.” Entering error mode will cause a visual or audible signal to be emitted to the operator, and/or cause one or more instructions to the reader to be displayed on the readout display. Similarly, if the control signal is equal to or above the threshold value (all systems go), will allow the user to continue with the assay procedure.

The reference spot 52 signal is then compared to one or more assay signals using a separate algorithm. This second algorithm is used to compare signal levels from at least two separate locations on the assay strip 16 and also to the control reference spot 52 signal. An emission intensity profile is then generated. This allows the instrument to compare the fluorescence intensities of the capture line 46, read line 48, and reference spot 52 in the determination of sample concentration.

A direct ratio of signal intensity can be used to determine analyte levels (FIG. 4). For example, the signal ratio of capture line: read line in a negative sample is 100:2 (=50); a medium positive would be 50:50, (=1); and a high positive would be 2:100 (=0.02). The two line signals may also be summed and this sum divided by the signal from the reference spot. This normalizes the signal so direct comparison from sample-to-sample can be made. Another algorithm possibility is a slope determination, based on the sample and read line peaks as the points that determine the line. The resulting line slope will vary inversely with sample concentration.

In at least one embodiment of the invention, the fluorescent intensity profile (intensity as a function of distance) determines a value of analyte concentration and will be presented on the screen display 64 for immediate use. The intensity profile and concentration values may then be stored in non-volatile memory of the electronic data storage unit 18 along with any test sample descriptive information entered by the user through a key pad 66 or screen display 64.

The foregoing description of preferred embodiments of the present invention has been provided for the purposes of illustration and description. It is not intended to be exhaustive or to limit the invention to the precise forms disclosed.

1. A portable device for reading of an immunochromatographic lateral flow diagnostic assay strip, comprising
a holder for positioning the assay strip in the device;
an excitation source for causing fluoresence of the assay strip;
a scanner for reading the assay strip by registering an intensity distribution of the fluorescence of the assay strip and converting the distribution into formatted data;
a data storage unit;
an input means for controlling operation of the device; and
a display for viewing a selected presentation of the data.

2. The device of claim 1 wherein the holder is a radial cassette having a plurality of assay strips positioned as spokes emanating from a central hub, and wherein the cassette is rotatable about the hub so that each individual assay strip may be separately positioned in relation to the scanner for registering the fluorescence of the individual assay strip.

3. The device of claim 2 wherein the cassette further comprises an input port for receiving a predetermined amount of a liquid sample for application to the assay strips and a distributor for dividing the liquid into predetermined portions and directing selected predetermined portions to selected ones of the assay strips.

4. The device of claim 1 wherein the excitation source is an ultraviolet light source positioned beneath the assay strip as it is being read and the photoelectric scanner is a photodiode array positioned above the assay strip.

5. The device of claim 2 wherein the cassette is removable from the device.

6. The device of claim 3 wherein the cassette is removable from the device.

7. The device of claim 2 wherein the assay strips may be removably inserted into the cassette.

8. The device of claim 3 wherein the assay strips may be removably inserted into the cassette.

9. The device of claim 5 wherein the assay strips may be removably inserted into the cassette.

10. The device of claim 6 wherein the assay strips may be removably inserted into the cassette.

11. A radial cassette comprising a circular cassette body and a plurality of holders for immunochromatographic lateral flow diagnostic assay strips positioned as spokes emanating from a central hub.

12. The cassette of claim 11 further comprising an input port for receiving a predetermined amount of a liquid sample for application to the assay strips and a distributor for dividing the liquid into predetermined portions and directing selected predetermined portions to selected ones of the assay strips.

13. The cassette of claim 11 wherein the assay strips are attached to the cassette.

14. The cassette of claim 12 wherein the assay strips are attached to the cassette.

15. A method for determining the amount of an analyte present in a sample comprising:
collecting the sample, wherein the sample is collected from the group comprising: a liquid sample and a diluted soil sample;
applying the sample to a cassette unit, wherein the cassette unit comprises a circular wheel and a sample splitter, and wherein the cassette unit is further capable of receiving a plurality of immunochromatographic assay strips;
contacting the sample to a plurality of immunochromatographic assay strips;
running a lateral flow assay along the immunochromatographic assay strip, wherein the running comprises allowing the sample to flow laterally along an assay strip, wherein the sample first contacts a conjugate comprised of assay reporter-detector pairs, wherein detectors in the pairs have an affinity to a target analyte, and assay reporters in the pairs are capable of being detected by a scanner; wherein the sample further contacts a capture line conjugate, the capture line conjugate comprised of a known concentration of analytes bound to the assay strip; and wherein the sample further contacts a read line conjugate, the read line conjugate comprising a known concentration of antibodies;
scanning the sample with a portable device capable of detecting the assay reporter presence at unique points on the assay strip.

16. The method of claim 15, and wherein the conjugate comprised of assay reporter-detector pairs further comprises fluorescent semiconducting nanocrystals coupled to antibodies specific for a target analyte.

17. The method of claim 15, wherein the conjugate comprised of assay reporter-detector pairs is further capable of binding to a target analyte, and wherein the reporter-detector pairs are capable of binding at the capture line if no analyte is bound to the reporter-detector pair, are further capable of binding at the read line where analyte is present.

18. The method of claim 15, wherein the device includes a spectrophotometric scanner coupled to an electronic data storage unit.

19. The method of claim 18, wherein the spectrophotometric scanner is further capable of detecting fluorescence emitted from the capture line, the read line, and the reference spot.

20. The method of claim 19 wherein the method further comprises the steps of: collecting the fluorescence emission output data from the spectrophotometric scanner;
and analyzing fluorescence emission output data.

21. The method of claim 20, wherein the analyzing fluorescence emission output data further comprises the step of: transmitting the fluorescence emission output data from electronic data storage unit.

22. The method of claim 21, wherein the method further comprises the steps of: measuring the emitted light from a reference spot with a predetermined emitted light signal, wherein the reference spot is at a unique address on the strip; measuring the emitted light from the read line and the capture line; comparing the fluorescence intensities of the emitted light from the signal emitted by the sample and the emitted light from the reference spot for each assay that is integrated into the cassette; comparing signal levels from at least two separate locations on the test strip, and the control signal; generating an emission intensity profile which allows the instrument to compare the fluorescence intensities of the capture line, read line, and reference spot; and determining the analyte concentration in the sample.
